(19) United States
(12) Patent Application Publication
(10) Pub. No.: US 2018/0099110 A1
(43) Pub. Date: Apr. 12, 2018

(54) ENDOTRACHEAL TUBE

(71) Applicant: Marco Mikhael, Hinsdale, IL (US)

(72) Inventor: Marco Mikhael, Hinsdale, IL (US)

(21) Appl. No.: 15/289,232

(22) Filed: Oct. 10, 2016

(51) Int. Cl. A61M 16/04

(52) U.S. Cl. CPC A61M 16/0459; A61M 2240/00; A61M 16/0447; A61M 16/044

(57) ABSTRACT

An improved endotracheal tube is disclosed. In particular, the disclosed endotracheal tube includes a distal end, which is adapted to be placed into a patient, and a proximal end, which is adapted to couple with a gas supply. The endotracheal tube includes an inflatable cuff disposed near the distal end of the tube, which exterior dimensions taper and narrow from the distal end towards the proximal end. In addition, the distal end of the endotracheal tube includes a second inflatable cuff that is adapted to dull the distal end of the endotracheal tube. In particular, the second inflatable cuff is adapted to dull the distal end to prevent or at least minimize irritation of the patient's mucosal tissue in the nose or throat.

### FIELD OF THE DISCLOSURE

The present disclosure generally relates to endotracheal tubes and more particularly relates to endotracheal tubes for oral and nasal intubation.

### BACKGROUND

Endotracheal intubation is a common medical procedure. Endotracheal tubes are used in many different types of procedures. For example, they are used as conduits for airway life support where patients have stopped breathing on their own due to cardiac arrest, respiratory distress, seizures, etc. They are also used during certain surgeries to provide respiratory support to patients while certain surgeries are conducted.

Endotracheal tubes generally comprise a long, somewhat flexible tube with an open distal end that is inserted into a patient's lungs, and a proximal end having an attachment allowing it to couple with a ventilating machine, which provides periodic measured drafts of air to the patient. To ensure that air flow is provided to the patient, an inflatable cuff is disposed on the exterior of the tube, and, after the tube is inserted, the cuff is inflated to provide an air tight seal within the patient's throat. The cuff is generally fed by a separate air pipe, which is referred to as a pilot, that is run along the inside or outside of the endotracheal tube directly to the cuff. An example of a standard prior art endotracheal tube is depicted in FIG. 1. In particular, the prior art endotracheal tube 30 comprises a tubular member 10 with a distal end 12 and a proximal end 14. The distal end 12 is beveled, meaning cut at an angle. An endotracheal tube adapter 16, which is a connector with a ventilating machine, is at the proximal end of the tubular member 10. The endotracheal tube adapter 16 is generally coupled to a gas source, such as an oxygen and air mixture, or an anesthetic gas mix, and provides treatment via the endotracheal tube 30. The tubular member 10 may comprise any suitable dimensions, and generally, a number of commonly used sizes ranging from those appropriate to neonatal patients, to those appropriate to very large patients, are available. The endotracheal tube 30 further comprises an inflatable cuff 20. The inflatable cuff 20 is adapted to be inserted into the trachea of the patient, past the patient's vocal cords 7. The inflatable cuff 20 is generally fed by its own inflation source using a separate tube 22. While the inflation tube 22 is shown as sitting outside of the tubular member 10, it is also commonly disposed inside the tubular member 10; i.e., in between an exterior layer of the tubular member 10 and an interior layer of the tubular member 10.

The general procedure for inserting an endotracheal tube is well understood, and illustrated in Prior Art FIG. 2. In particular, a medical professional will obtain an endotracheal tube in step 102, and insert the distal end of the endotracheal tube into the patient's mouth or nose, depending on whether the intubation will be oral or nasal in step 104. The medical professional will then guide the tube until it is properly positioned in step 106, as discussed below. Generally, prior art endotracheal tubes will have one or more guidelines that a professional can position within the part of the patient, which will vary based on whether the insertion is oral or nasal, that the professional can see. Once the tube is properly positioned within the patient, the professional will cause the cuff to be inflated so that an airtight seal is provided in step 108. The professional will then secure the endotracheal tube to the patient in step 110; for example, the professional may tape the tube in place.

One type of popular endotracheal tube is typically made of soft biomaterials. The most common biomaterial is polyvinyl chloride with a plasticizer added, but other materials are also used or are in development. Endotracheal tubes made of, for example, a soft polyvinyl chloride, can be difficult to manipulate and insert into a patient's trachea (windpipe). Accordingly, a malleable stylette is often placed into a central lumen of an endotracheal tube to provide more rigidity to the tube and allow the caregiver to have greater control in manipulating the tube into place during insertion. The stylette can be, for example, a malleable rod of plastic, or a plastic coated metal. Normally, the stylette is inserted along the length of the endotracheal tube's central lumen to a point just before the endotracheal tube ends. After the endotracheal tube is inserted into the trachea, the stylette is manually removed.

Another type of endotracheal tube is typically made of a more rigid PVC (polyvinyl chloride). This type of tube has the advantage of not requiring a malleable stylette, and the extra procedures and cost associated therewith. However, the added rigidity can make insertion of the tube more difficult.

To accommodate patients having differently sized trachea, endotracheal tubes are generally provided in different diameters to permit selection of the proper size tube for the patient. To provide the most unobstructed passageway to the lungs of a particular patient, generally, the largest possible diameter tracheal tube acceptable to the patient is utilized. In certain circumstances, where oral intubation is not possible, nasal insertion of the tracheal tube can be required. In both oral and nasal intubation, the edge of the distal end of the endotracheal tube can irritate a patient's mucosal tissue. The hardness of the material of the endotracheal tube factors into the amount of irritation that a patient might experience. Hardness can be measured, for example, using an indentation hardness measurement, such as, for example, Shore durometer.

Proper insertion of an endotracheal tube is vital, as harm to the patient can result if the tube is not inserted far enough, or too far. If the endotracheal tube extends beyond the carina; i.e., the point at which the trachea branches into the lungs, and into the patient's right mainstem bronchus, the bronchial intubation can result in injury, which is generally referred to as Right Mainstem Bronchial Intubation (RMSBI). Alternatively, if the tube is inserted so that the cuff is not entirely past the patient's vocal cords, when the cuff is inflated, the patient's vocal cords can be damaged. Both issues are complicated by endotracheal tubes coming in a number of fixed sizes (both in terms of total length, and the positioning of the cuff relative to the distal end), and patients not falling into a small number of sizes with regards to these dimensions.

Accordingly, there is a need for an endotracheal tube that allows for insertion without irritating a patient's mucosal tissue in the nose, mouth, or the trachea. There is also a need for an endotracheal tube that allows for insertion without damaging the patient's vocal cords, and diminishes the risk of RMSBI.

### Objects of the Disclosed System, Method, and Apparatus

Accordingly, it is an object of this disclosure to provide an improved endotracheal tube.

Another object of the disclosure is to provide an improved endotracheal tube that minimizes irritation to a patient's mucosal tissue in the nose, mouth and throat.

Another object of the disclosure is to provide an improved endotracheal tube that can be used for both nasal and oral intubation.

Another objection of the disclosure is to provide an improved endotracheal tube that allows for precise positioning of the cuff relative to the patient's vocal cords.

Another object of the disclosure is provide an improved endotracheal tube that reduces the risk of RMSBI.

Another object of the disclosure is to provide an improved endotracheal tube that reduces the risk of irritating a patient's vocal cords.

Another object of the disclosure is to provide an endotracheal tube that incorporates a second inflatable cuff on the distal end of the tube.

Another object of the disclosure is to provide an endotracheal tube with an inflatable cuff that tapers from the end of the cuff disposed towards the distal end of the tube toward the end of the cuff disposed towards the proximal end of the tube.

Other advantages of this disclosure will be clear to a person of ordinary skill in the art. It should be understood, however, that a system or method could practice the disclosure while not achieving all of the enumerated advantages, and that the protected disclosure is defined by the claims.

### SUMMARY OF THE DISCLOSURE

An improved endotracheal tube is disclosed. The disclosed endotracheal tube comprises a tubular member, which as disclosed herein, can be of any suitable material and dimensions. The tubular member includes a proximal end and a distal end. The proximal end is adapted to couple with a ventilating machine, and may include an adapter as disclosed herein. The distal end includes an opening that is adapted to be disposed past a patient's vocal cords. An inflatable cuff is disposed laterally along the tubular member of the endotracheal tube near the distal end, with the inflatable cuff being adapted to occlude the patient's trachea when inflated so as to prevent air from escaping up the patient's trachea when inflated. The inflatable cuff has a far side, disposed toward the distal end of the tubular member, and a near side, disposed toward the proximal end of the tubular member. The circumference of the exterior surface of the far side is greater than the circumference of the near side. Preferably, the circumference of the near side of the inflatable cuff when inflated is substantially the same as the circumference of the tubular member, while the circumference of the far side must be such as to occlude the trachea of the patient, and prevent air from escaping up the patient's trachea. In addition, a second inflatable cuff is disposed on the distal end of the tubular member. In particular, the second inflatable cuff can be rounded, engulfing the distal tube edge in such manner as to prevent direct contact of the distal end of the endotracheal tube with the patient's tissue, or can be made of a material having a lower Shore hardness score than the material comprising the tubular member.

A method of inserting an endotracheal tube into a patient is also disclosed. In particular, the method comprises the steps of obtaining an endotracheal tube comprising a tubular member having a proximal end and a distal end. The obtained endotracheal tube has a cuff disposed laterally thereon near the distal end of the endotracheal tube. The endotracheal tube has a far end, disposed towards the distal end of the endotracheal tube, and a near end, disposed toward the proximal end of the endotracheal tube. When inflated, the exterior circumference of the far end of the cuff is greater than the exterior circumference of the near end of the cuff. The distal end of the endotracheal tube also has a second inflatable cuff disposed thereon, with the second inflatable cuff being adapted to dull the distal end of the endotracheal tube when inflated. After obtaining the endotracheal tube, a medical practitioner inserts the distal end of the endotracheal tube into a patient, and then positions the tube so that the cuff is properly positioned within the patient. Preferably, the cuff is positioned so that it is placed clear of the patient's vocal cords, although the design of the disclosed inflatable cuff is adapted to minimize any discomfort or harm suffered by the patient if the cuff is placed proximate to the patient's vocal cords. Finally, the medical practitioner secures the endotracheal tube within the patient by, for example, taping the tube in position.

### BRIEF DESCRIPTION OF THE DRAWINGS

Although the characteristic features of this disclosure will be particularly pointed out in the claims, the invention itself, and the manner in which it may be made and used, may be better understood by referring to the following description taken in connection with the accompanying drawings forming a part hereof, wherein like reference numerals refer to like parts throughout the several views and in which:

FIG. 1 is a perspective view of a prior art endotracheal tube inserted orally into a patient;

FIG. 2 is a listing of steps that a medical professional may follow to insert an endotracheal tube into a patient;

FIG. 3 is a perspective view of an endotracheal tube constructed in accordance with an aspect of this disclosure;

FIG. 4 is a perspective view of an endotracheal tube constructed in accordance with a second aspect of this disclosure;

FIG. 4A is aclose up perspective view of the cuff of an endotracheal tube constructed in accordance with an aspect of this disclosure;

FIG. 5 is a perspective view of an endotracheal tube constructed in accordance with a third aspect of this disclosure;

FIG. 5a is a close up perspective view of a deflated second cuff of an endotracheal tube constructed in accordance with an aspect of this disclosure;

FIG. 5b is a close up perspective view of an inflated second cuff of an endotracheal tube constructed in accordance with an aspect of this disclosure; and

FIG. 6 is a perspective view of an endotracheal tube constructed in accordance with a fourth aspect of this disclosure.

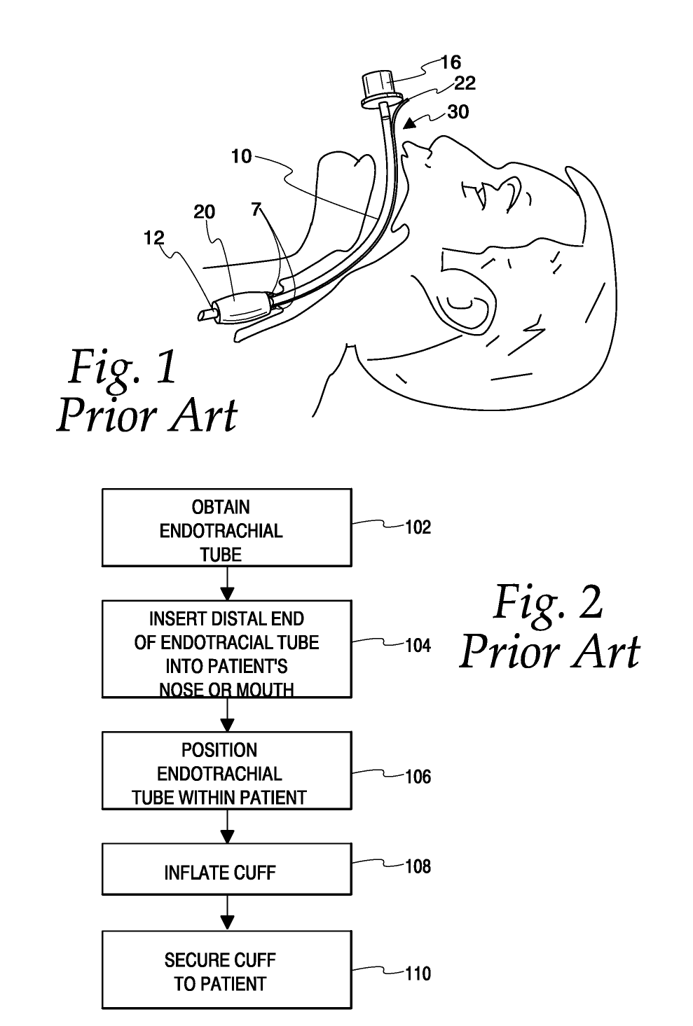

### DETAILED DESCRIPTION

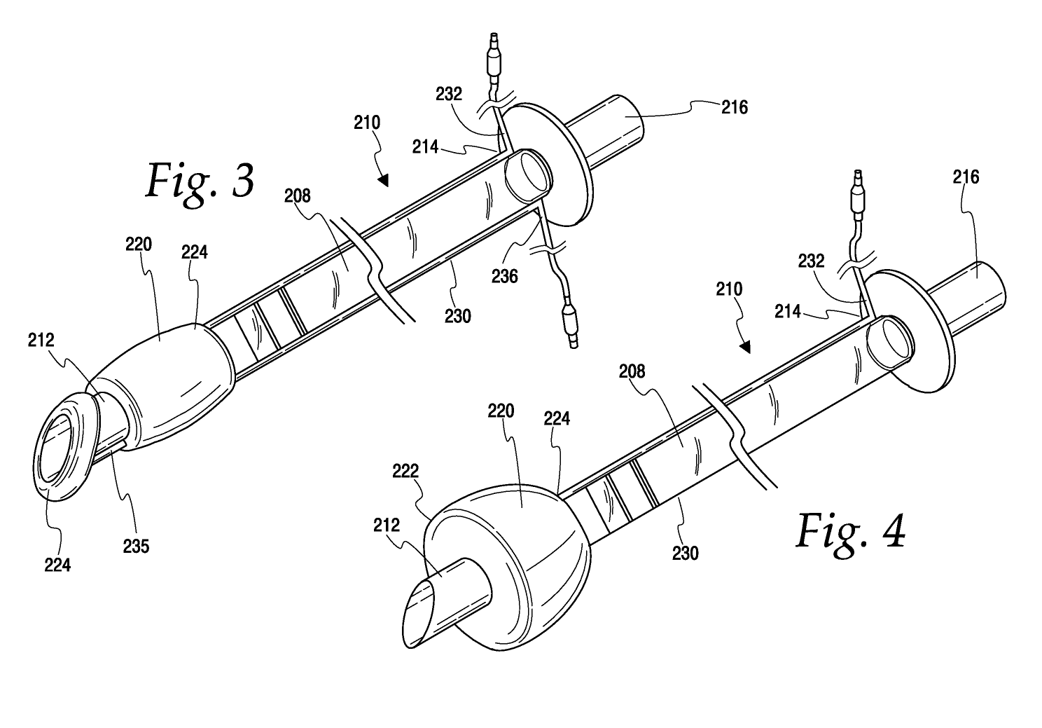

Turning to FIG. 3, a first aspect of the disclosed endotracheal tube is illustrated. In particular, an endotracheal tube device 230 comprises a thin walled, elongated tubular member 210 having a proximal end 214 and an open distal end 212. An inflatable cuff 220 is coupled to the tubular member 210 near the distal end 212 of the endotracheal tube. The inflatable cuff 220 is inflated by pilot 232, and can be inflated by, for example, a syringe (not shown) inserted into an opening at the end of the pilot 208. The endotracheal tube is sufficiently long so that, when the tube is inserted into a patient's trachea, the distal end and inflatable cuff are located below the patient's vocal cords and the proximal end is disposed in the patient's mouth or nose, depending on whether the endotracheal tube is designed for oral or nasal use.

The endotracheal tube 200 also includes an adapter 216 at the proximal end 214 of the tubular member 210. The adapter 216 can be coupled to a gas source, such as an assisted ventilation device (not pictured), to provide treatment via the endotracheal tube 230. The treatment can be, for example, a mixture of oxygen and air with or without anesthetic, or other substances. The adapter 216 can be internally or externally coupled to the proximal end 214 of the tubular member 210 so that the adapter 216 can be coupled to a ventilating device.

The endotracheal tube 230 and its constituent components can comprise any suitable dimensions. Generally, the dimensions will be sized as appropriate to the patient; i.e., the dimensions of the endotracheal tube 230 will be smaller if the tube is adapted for use on a neonate that if it is adapted for use with an adult male of normal size.

Similarly, the tubular member 210 can be constructed of any suitable material, such as, for example, a flexible medical grade plastic material. Suitable materials include polyvinyl chloride, silicone elastomer, polyurethanes, etc. Similarly, the cuff 220 can also be constructed of any suitable material, such as, for example, a polyurethane.

The disclosed endotracheal tube 200 also incorporates a second cuff 224 disposed at the distal end 212 of the tubular member 210. The second cuff is inflated using a second pilot 235 having an opening 236. The second cuff 224 is adapted to blunt the end of the endotracheal tube 200. When inflated, the second cuff 224 will generally comprise a rounded lip about the distal end 212 of the endotracheal tube 230. The additional softened material comprising the second cuff 224 blunts the distal end 212 of the tubular member 210, so that, for example, when it is inserted into the patient's nose, it will not irritate, or cut the mucosal tissue therein, or at least, it will reduce the irritation of the patient's nasal mucosal tissue. Similarly, rounding the second cuff 224 blunts the distal end 212 of the tubular member 210 so that, if inserted orally, it will minimize the irritation of the mucosal tissue in the patient's trachea. The second cuff 224 may be made of a material that is less hard than the material comprising the tubular member 210. For example, the second cuff 224 can be made of a soft elastomer or polyurethane while the tubular member 210 is made of a semi-rigid polyvinyl chloride.

The second cuff 224 will generally be inflated by an air syringe using the second pilot 236 through an opening on the end of the second pilot 236.

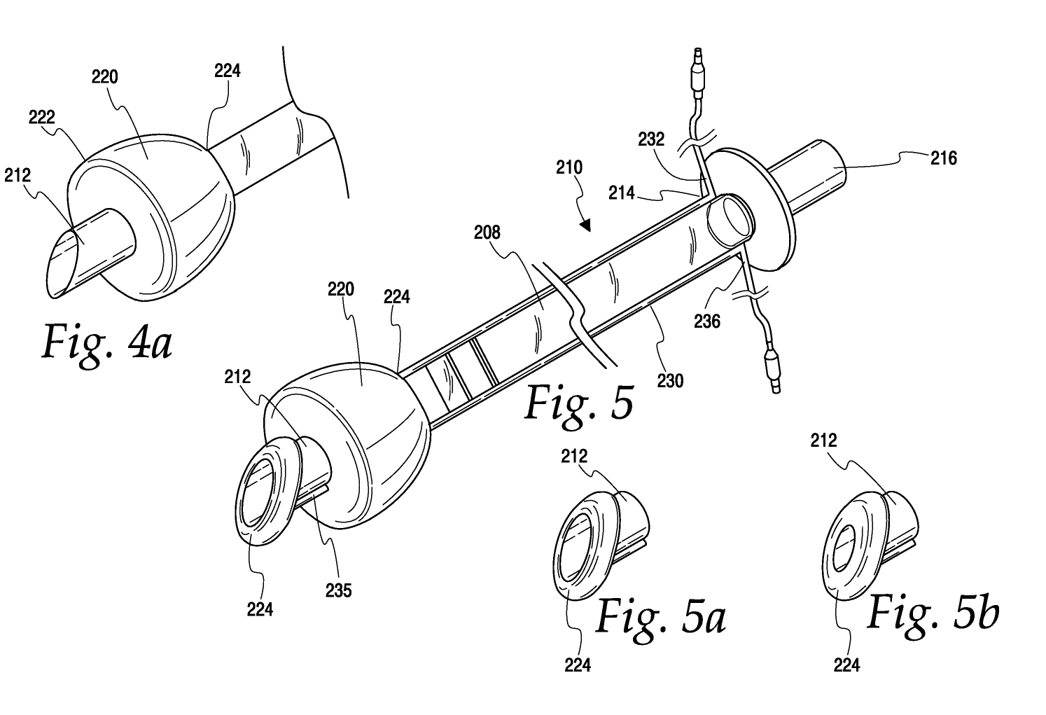

Turning to FIG. 4, a second aspect of the disclosed endotracheal tube is illustrated. The tubular member 210, distal end 212, proximal end 214 and adapter 216 are identical to the embodiment discussed in FIG. 3. However, the inflatable cuff 220 differs substantially from the embodiment of FIG. 3. In particular, the inflatable cuff 220 includes a far end 222, which is closest to the distal end 212 of the tubular member 210, and a near end 224, which is closest to the proximal end 214 of the tubular member 210. When inflated, the far end 222 of the inflatable cuff 220 has a greater diameter than the near end 224 of the inflatable cuff. This can be accomplished by changing the dimensions of the material of the inflatable cuff as depicted in FIG. 4A.

The modified cuff 220 of this embodiment offers the substantial embodiment causing less irritation to a patient's vocal cords if the cuff 220 is inflated when disposed at a location that is not clear of the patient's vocal cords. The taper in the cuff's exterior dimensions allows the cuff 220 to be inflated even if the cuff 220 is not entirely clear of the patient's vocal cords because the dimension of the near end 224 of the cuff is narrower than the gap of the client's vocal cords (not shown), or at worst, it will minimally spread the client's vocal cords, rather than the significant spreading and contact that patients suffered through with prior art cuffs.

FIG. 5 is identical to the embodiment of FIG. 3 except that, in addition to the second cuff 224 at the distal end 212 of the tubular member 210, it also incorporates the tapered cuff 220 of FIG. 4. Accordingly, it provides all of the advantages of the embodiments of FIGS. 3 and 4.

FIG. 5a depicts the second inflatable cuff 224 disposed near the distal end 212 of the endotracheal tube when the second inflatable cuff 224 is not inflated. FIG. 5b depicts the second inflatable cuff 224 disposed near the distal end 212 of the endotracheal tube when the second inflatable cuff 224 is inflated.

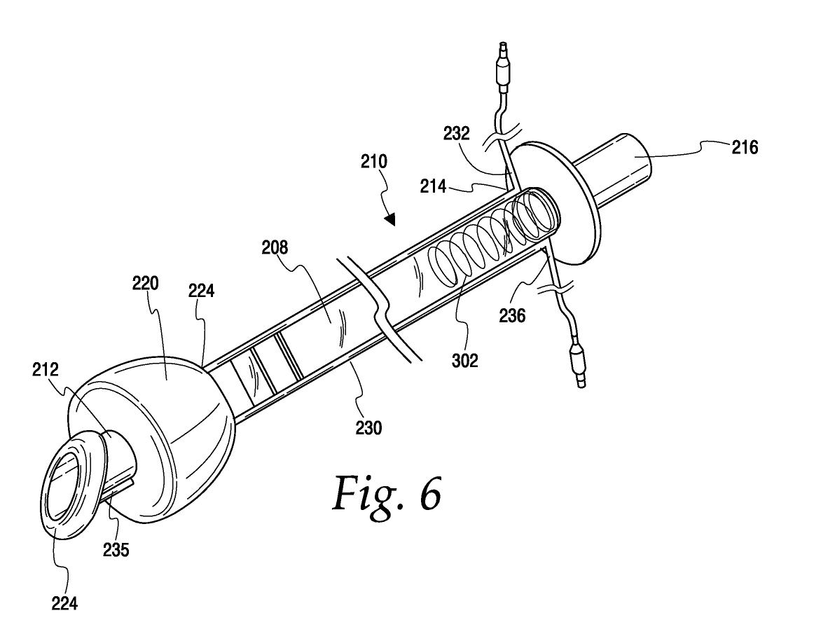

FIG. 6 is identical to the embodiment of FIG. 5 with the addition of a spirally-wound coil 302 that is disposed between an inner and outer layer of the tubular member 210. The spirally-wound coil 302 is fabricated of a shape-memory flexible material that is adapted to return to or recover its original shape if compressed. The particular shape-memory material could be any suitable material, including a shape-memory alloy or polymer.

The foregoing description of the disclosure has been presented for purposes of illustration and description, and is not intended to be exhaustive or to limit the disclosure to the precise form disclosed. The description was selected to best explain the principles of the present teachings and practical application of these principles to enable others skilled in the art to best utilize the disclosure in various embodiments and various modifications as are suited to the particular use contemplated. It is intended that the scope of the disclosure not be limited by the specification, but be defined by the claims set forth below. In addition, although narrow claims may be presented below, it should be recognized that the scope of this invention is much broader than presented by the claim(s). It is intended that broader claims will be submitted in one or more applications that claim the benefit of priority from this application. Insofar as the description above and the accompanying drawings disclose additional subject matter that is not within the scope of the claim or claims below, the additional inventions are not dedicated to the public and the right to file one or more applications to claim such additional inventions is reserved.

What is claimed is:

1. An endotracheal tube adapted for ventilating a patient comprising:
a) a tubular member;
b) a proximal end of the tubular member adapted to couple with a ventilating machine;
c) a distal end of the tubular member having an opening that is adapted to be disposed past the patient's vocal cords;
d) an inflatable cuff disposed near the distal end, the inflatable cuff disposed laterally along the tubular member and being adapted to occlude the patient's trachea when inflated so as to prevent air from escaping up the patient's trachea past the vocal cords wherein the inflatable cuff has a far end disposed towards the distal end of the endotracheal tube, and a near end disposed toward the proximal end of the endotracheal tube and wherein the circumference of the inflatable cuff when inflated at the far end is greater than the circumference of the inflatable cuff when inflated at the near end; and
e) a second inflatable cuff disposed at the proximal end of the endotracheal tube, the second inflatable cuff being adapted to dull the proximal end of the endotracheal tube.

2. The endotracheal tube of claim 1 wherein the second inflatable cuff is rounded.

3. The endotracheal tube of claim 1 wherein the second inflatable cuff is made of a material having a lower hardness measure than the tubular member.

4. The endotracheal tube of claim 1 wherein the tubular member is of a length adapted for use in a neonate.

5. The endotracheal tube of claim 1 wherein the tubular member is of a length adapted for use in a large patient.

6. The endotracheal tube of claim 1 wherein the tubular member is constructed of polyvinyl chloride.

7. The endotracheal tube of claim 3 wherein the second inflatable cuff is constructed of an elastic isomer.

8. A method of inserting an endotracheal tube into a patient, the method comprising the steps of:
a) obtaining an endotracheal tube comprising a tubular member having a proximal end and a distal end, the endotracheal tube further having an inflatable cuff disposed near the distal end, the inflatable cuff disposed laterally along the tubular member and having a far end disposed towards the distal end of the tubular member and a near end disposed towards the proximal end of the tubular member wherein, when inflated, the far end has a greater circumference than the near end, the distal end of the endotracheal tube further having a second inflatable cuff disposed thereon, the second inflatable cuff being adapted to dull the distal end of the endotracheal tube;
b) inflating the second inflatable cuff;
c) inserting the distal end of the endotracheal tube into a patient;
d) inflating the inflatable cuff when the endotracheal tube is properly positioned within the patient; and
e) securing the endotracheal tube within the patient.
